(19) United States
(12) Patent Application Publication
(10) Pub. No.: US 2006/0154329 A1
(43) Pub. Date: Jul. 13, 2006

(54) Expression system

(76) Inventors: Irene Mary Horne, New South Wales (AU); Robyn Joyce Russell, Australian Capital Territory (AU); John Graham Oakeshott, Australian Capital Territory (AU); Tara Deane Sutherland, Watson, ACT (AU)

Correspondence Address: COOPER & DUNHAM, LLP, 1185 AVENUE OF THE AMERICAS, NEW YORK, NY, 10036, US

(21) Appl. No.: 10/522,427

(22) PCT Filed: Jul. 28, 2003
(86) PCT No.: PCT/AU03/00951

§ 371 Date: Sep. 2, 2005

(30) Foreign Application Priority Data
Jul. 26, 2002 (AU) 2002950473

(51) Int. Cl. C12P 21/06; C07H 21/04; C12N 1/21; C07K 14/32

(52) U.S. Cl. 435/69.1; 435/471; 435/252.31; 530/326; 536/23.7

(57) ABSTRACT

The present invention provides a recombinant polynucleotide, the polynucleotide comprising a first and a second sequence, the first sequence encoding a signal peptide comprising a TAT signal and a Sec avoidance signal and the second sequence encoding a heterologous protein. The sequence of the signal peptide is M-X₁—K/R—X₂—K/R—X₃—RR—X₄—K/R-A in which X₁ is a sequence of 0 to 10 amino acids, X₂ is a sequence of 0 to 3 amino acids, X₃ is a sequence of 0 to 10 amino acids and X₄ is a sequence of 15 to 24 amino acids in which at least 75% up to about 90% of the residues are hydrophobic.

The present invention relates to a modified expression system for the transport and secretion of polypeptides. More specifically it relates to expression of proteins which readily fold in the cytoplasm.

### BACKGROUND OF THE INVENTION

There is increasing interest in expression systems which enable bacterial production of recombinant proteins. In particular there is a need for systems which provide for high yield of recombinant proteins in culture supernatants particularly for agricultural and industrial uses. Recently, secretion of high yields of recombinant protein have been obtained using Brevibacillus.

In bacteria, following expression of a protein, transmembrane translocation of the protein can proceed by a number of routes depending on the nature of both the targeting signal and the substrate. In general, most proteins destined for export are synthesised with N-terminal extensions, called signal peptides. Signal peptides consist of short stretches of amino acids which, after protein delivery to the subcellular fraction, are removed. In bacteria two secretion pathways have been identified. The best characterised system is the Sec system for the general secretion of proteins. In this transport system, proteins are threaded through the membrane and correct folding occurs after transport.

The second system recently identified by Berks (1996) is the TAT system (for twin-arginine transfer peptide-dependent protein translocase). This system is so-called because the signal peptide generally contains the distinctive (S/T)RRXFLK motif. Proteins transported by this system are folded prior to translocation. Therefore, proteins of this type are toxic to bacteria if attempts are made to transport them through the membrane with a Sec secretion signal, significantly limiting the production and recovery of the enzyme in these systems.

In general, cytoplasmic proteins readily fold in the cytoplasm of heterologous hosts. Therefore it would be predicted that this group of proteins may not be readily secreted by the Sec system. β-galactosidase is a classic example of this. This particular enzyme from E. coli is a large protein with 120 kDa subunits and known to not pass through the cytoplasmic membrane when attached to a Sec-type secretion peptide (Manoil & Beckwith, 1985; Bassford et al., 1979). An alternative system is required to facilitate recovery of proteins that readily fold in the cytoplasm.

One example of recombinant proteins which are required in high yield are enzymes which can be used to degrade organic pollutants.

Microorganisms are involved in the degradation of many organic compounds and are the principal agents for the biodegradation and recycling of organic matter. The degradation of organic compounds by microorganisms is primarily due to the action of various enzymes produced by the microorganism. The role of these enzymes in degrading organic pollutants has been investigated and a number of enzymes derived from microorganisms have been identified that may be useful in assisting in bioremediation and the clean up of environmental pollutants and toxic compounds such as organophosphate pesticides.

Residues of organophosphate insecticides are undesirable contaminants of the environment and a range of commodities. Areas of particular sensitivity include contamination of soil, irrigation tailwater that is re-cycled, used by irrigators downstream or simply allowed to run off-farm, and residues above permissible levels in meat and horticultural exports. Poisoning with organophosphates presents a problem for agricultural workers that are exposed to these chemicals, as well as military personnel exposed to organophosphates used in chemical warfare. Furthermore, the stockpiling of organophosphorus nerve agents has resulted in the need to detoxify these stocks. Bioremediation strategies are therefore required for eliminating or reducing these organophosphate residues and/or stockpiles.

One proposed strategy involves the use of enzymes capable of immobilising or degrading the organophosphate residues. Such enzymes may be employed, for example, in bioreactors through which contaminated water could be passed, or in washing solutions after post-harvest disinfestation of fruit, vegetables or animal products to reduce residue levels and withholding times. Suitable enzymes for degrading organophosphate residues include OP hydrolases from bacteria (Mulbry, 1992; Mulbry and Kearney, 1991; Cheng et al., 1999; U.S. Pat. No. 5,484,728; U.S. Pat. No. 5,589,386; Horne et al., 2002; PCT/AU02/00594), vertebrates (Wang et al., 1993; 1998; Gan et al, 1991; Broomfield et al., 1999) and OP resistant insects (PCT/AU95/00016 and PCT/AU96/00746).

The most thoroughly studied OP degrading enzyme is bacterial organophosphate dehydrolase (OPD), which is encoded by identical genes on dissimilar plasmids in both Flavobacterium sp. ATCC 27551 and Brevundimonas diminuta MG (Harper et al., 1988; Mulbry and Karns, 1989). OPD is a homodimeric protein that is capable of hydrolysing a wide range of phosphate triesters (both oxon and thion OPs) with impressive kinetics (Dumas et al., 1989a, b). Its reaction mechanism directly or indirectly involves metal ions, preferably Zn⁺⁺. OPD has no detectable activity with phosphate monoesters or diesters (Dumas et al., 1989a, b; 1990).

OPD homologues (phosphotriesterase homology proteins, or PHPs) have been identified in the genomes of Escherichia coli (ePHP), Mycobacterium tuberculosis (mtPHP) and Mycoplasma pneumoniae (mpPHP), although only ePHP has been tested for phosphotriesterase activity (Scanlan and Reid, 1995; Buchbinder et al., 1998). OPD homologues have also been identified in vertebrates (Davies et al., 1997), although their function in these organisms is unknown.

OPD/OpdA have been identified as important enzymes that may assist in the bioremediation and dean up of environmental organophosphorus pollutants. Microorganisms such as bacteria should provide an important production tool for OPD/OpdA and various systems have been employed to increase the production and output of this enzyme. However expression systems are not available to provide sufficient quantities of this enzyme to meet the growing demand.

WO 02/22667 claims methods of producing polypeptides using the signal sequences from PhoD or LipA. This reference, however, provides no examples of production of polypeptides using a LipA signal sequence.

The present inventors have developed a novel expression system comprising a modified signal peptide which includes a Sec avoidance signal. It was found that this Sec avoidance signal was of critical importance for the production of polypeptides. This expression system has enabled the production of OpdA and other cytoplasmic proteins in high yields from Brevibacillus.

### SUMMARY OF THE INVENTION

In a first aspect the present invention provides a recombinant polynucleotide, the polynucleotide comprising a first and a second sequence, the first sequence encoding a signal peptide comprising a TAT signal and a Sec avoidance signal and the second sequence encoding a heterologous protein, wherein the sequence of the signal peptide is
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

In a second aspect the present invention provides a signal peptide, the signal peptide having the sequence
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

In a third aspect the present invention provides a method of producing a heterologous polypeptide from a host cell comprising a TAT translocation system, the method comprising:

(i) transforming the host cell with a DNA sequence encoding the heterologous polypeptide and a signal peptide wherein the signal peptide comprises a TAT signal and a Sec avoidance signal wherein the sequence of the signal peptide is
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.
   (ii) culturing the host cell under conditions which allow expression of the heterologous polypeptide; and
   (iii) recovering the heterologous polypeptide secreted from the host cell via the TAT translocation system.

Preferably, the host cell is Bacillus sp. in particular a strain that produces little or no exoproteases such that degradation of the expressed protein is minimised. More preferably, the host cell is Bacillus choshinensis, Bacillus brevis, Bacillus subtilis, Bacillus licheniformis, or Bacillus megatorium. It is most preferred that the host cell is. Brevibacillus sp, particularly Bacillus choshinensis. It is further preferred that the host cell is as described in U.S. Pat. No. 4,946,789.

In a further preferred embodiment the heterologous polypeptide is a polypeptide which readily folds in the cytoplasm. In particular embodiments the polypeptide is OpdA.

In a fourth aspect, the present invention provides a substantially purified polypeptide produced according to the method of the first aspect.

### DETAILED DESCRIPTION OF THE INVENTION

As discussed above the present inventors have developed a novel expression system comprising a modified signal peptide which includes a Sec avoidance signal. It was found that this Sec avoidance signal was of critical importance for the production of polypeptides. This expression system has enabled the production of OpdA and other cytoplasmic proteins in high yields from Brevibacillus.

Accordingly, in a first aspect the present invention provides a recombinant polynucleotide, the polynucleotide comprising a first and a second sequence, the first sequence encoding a signal peptide comprising a TAT signal and a Sec avoidance signal and the second sequence encoding a heterologous protein, wherein the sequence of the signal peptide is.
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

In a second aspect the present invention provides a signal peptide, the signal peptide having the sequence
M-X₁—K/R—X₂—K/R—X₃—RR—X₄—K/R-A
in which
X₁ is a sequence of 0 to 10 amino adds;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

In a third aspect the present invention provides a method of producing a heterologous polypeptide from a host cell comprising a TAT translocation system, the method comprising:

(i) transforming the host cell with a DNA sequence encoding the heterologous polypeptide and a signal peptide wherein the signal peptide comprises a TAT signal and a Sec avoidance signal wherein the sequence of the signal peptide is
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.
   (ii) culturing the host cell under conditions which allow expression of the heterologous polypeptide; and
   (iii) recovering the heterologous polypeptide secreted from the host cell via the TAT translocation system.

Preferably, the host cell is Bacillus sp. in particular a strain that produces little or no exoproteases such that degradation of the expressed protein is minimised. More preferably, the host cell is Bacillus choshinensis, Bacillus brevis, Bacillus subtilis, Bacillus licheniformis, or Bacillus megatorium. It is most preferred that the host cell is Brevibacillus sp, particularly Bacillus choshinensis. It is further preferred that the host cell is as described in U.S. Pat. No. 4,946,789.

In a further preferred embodiment the heterologous polypeptide is a polypeptide which readily folds in the cytoplasm. In particular embodiments the polypeptide is OpdA.

In a fourth aspect, the present invention provides a substantially purified polypeptide produced according to the method of the first aspect.

In a preferred embodiment of the present invention X₁ is a sequence of 0 to 5 amino acids, and is preferably 0. It is preferred that X₂ is a sequence of 0 or 1 amino acid, preferably 0. It is preferred that X₃ is a sequence of 0 to 5 amino acids, preferably 0.

It is preferred that X₄ is a sequence of at least 20 amino acids of which at least 18 are hydrophobic amino acids. It is preferred X₄ is 23 amino acids.

In order to avoid any doubt as used herein the term “hydrophobic amino acids” means an amino acid selected from the group consisting of isoleucine, leucine, alanine, valine, glycine, phenylalanine and proline.

In a further preferred embodiment the sequence of the signal peptide is MKKRRVVNSVLLLLLLASALALTVAPMAKA (SEQ ID No:1).

While it is believed that the concept of signal sequences including a TAT signal are well known for the sake of clarity a number of such sequences are set out in Table 1.

TABLE 1

A list of TAT sequences cited in Berks (1996). The
signature “twin arginine” motif is in bold type.

| | |
|---|---|
| MEARMTGRRKVTRRDAMADAARAVGVACLGGFSLAALVRTASPVDA | (SEQ No:2) |
| MSRSAKPQNGRRRFLRDVVRTAGGLAAVGVALGLQQQTARA | (SEQ No:3) |
| MTWSRRQFLTGVGVLAAVSGTAGRVVA | (SEQ No:4) |
| MDRRRFLTLLGSAGLTATVATAGTAKA (SEQ No:5) | |
| MSEKDKMITRRDALRNIAVVVGSVATTTMMGVGVADA | (SEQ No: 6) |
| MQIVNLTRRGFLKAACVVTGGALISIRMTGKAVA | (SEQ No :7) |
| MNNEETFYQAMRRQGVTRRSFLKYCSLAATSLGLGAGMAPKIAWA | (SEQ No:8) |
| MSTGTTNLVRTLDSMDFLKMDRRTFMKAVSALGATAFLGTYQTEIVNA | (SEQ No:9) |
| MKCYIGRGKNQVEERLERRGVSRRDFMKFCTAVAVAMGMGPAFAPKVAEA | (SEQ No:10) |
| MNRRNFIKAASCGALLTGALPSVSHA | (SEQ No:11) |
| MSHADEHAGDHGATRRDFLYYATAGAGTVAAGAAAWTLVNQMNP | (SEQ No:12) |
| MTQISGSPDVPDLGRRQFMNLLTFGTITGVAAGALYPAVKYLIP | (SEQ No:13) |
| MDRRTFLRLYLLVGAAIAVAPVIKPALDYVGY | (SEQ No:14) |
| MTKLSGQELHAELSRRAFLSYTAAVGALGLCGTSLLAQGARA | (SEQ No:15) |
| MTLTRREFIKHSGIAAGALVVTSAAPLPAWA | (SEQ No:16) |
| MTISRRDLLKAQAAGIAAMAANIPLSSQAPA | (SEQ No:17) |
| MSEALSGRGNDRRKFLKMSALAGVAGVSQAVG | (SEQ No:18) |
| MKTKIPDAVLAAEVSRRGLVKTTAIGGLAMASSALTLPFSRIAHA | (SEQ No:19) |
| MSNFNQISRRDFVKASSAGAALAVSNLTLPFNVMA | (SEQ No:20) |
| MSISRRSFLQGVGIGCSACALGAFPPGALA | (SEQ No:21) |
| MKTVLPSVPETVRLSRRGFLVQAGTITCSVAFGSVPA | (SEQ No:22) |
| MGRLNRFRLGKDGRREQASLSRRGFLVTSLGAGVMFGFARPSSA | (SEQ No:23) |
| MSDKDSKNTPQVPEKLGLSRRGFLGASAVTGAAVAATALGGAVMTRESWA | (SEQ No:24) |
| MESRTSRRTFVKGLAAAGVLGGLGLWRSPSWA | (SEQ No:25) |
| MSLSRRQFIQASGIALCAGAVPLKASA | (SEQ No:26) |
| MTLNRRDFIKTSGAAVAAVGILGFPHLAFG | (SEQ No:28) |
| MTDSRANRADATRGVASVSRRRFLAGAGLTAGAIALSSMSTSASA | (SEQ No:28) |

As will be understood by those skilled in this area the method of the present invention is suitable for expression of a number of cytoplasmic proteins, including but not limited to, lipases, proteases, esterases and enzymes involved in carbohydrate metabolism.

As is well known in the art signal peptidases remove signal peptides from secretory proteins. Signal peptidases such as signal peptidase I (SPase I) cleave the signal peptide at specific sites such as cleaving between contiguous alanine residues. In the present invention the C-terminal residue of the signal peptide is alanine. Accordingly, this signal peptide is adapted for use with a polypeptide which includes an alanine residue at the N-terminal of the mature protein.

In a fourth aspect, the present invention provides a substantially purified polypeptide produced according to the method of the first aspect.

In, another preferred embodiment the polynucleotide encoding the mature polypeptide has a sequence selected from:
(i) a sequence of nucleotides shown in SEQ ID NO:29 from nucleotide 85 to 1155;
(ii) a sequence that hybridises to SEQ ID NO:29 from nucleotide 85 to 1155 under conditions of high stringency; and
(iii) a sequence which is greater than 90% identical to SEQ ID NO:29 from nucleotide 85 to 1155.

Preferably the mature heterologous polypeptide expressed by the method of the third aspect comprises the sequence provided in SEQ ID NO:30 from residue 29 to 384; or a polypeptide which is greater than 90% identical to the sequence provided in SEQ ID No:30. More preferably, the polypeptide is at least 95% identical to the sequence provided in SEQ ID No:30, even more preferably at least 97% identical, and even more preferably at least 99% identical to the sequence provided in SEQ ID No:30.

The present invention also provides a suitable vector for the replication and/or expression of the polynucleotide. The vectors may be, for example, a plasmid or phage vector provided with an origin of replication, and preferably a promoter for the expression of the polynucleotide and optionally a regulator of the promoter. The vector may contain one or more selectable markers, for example an ampicillin resistance gene in the case of a bacterial plasmid. The vector may be used in vitro, for example to transfect or transform a host cell.

In a further aspect, the present invention relates to the transformed host cell of the third aspect.

In a preferred embodiment the host cell is a Brevibacillus sp., preferably Bacillus choshinensis. Preferably, the host cell produces little or no exoproteases such that degradation of the expressed protein is minimised. It is further preferred that the host cell is as described in U.S. Pat. No. 4,946,789. Such cells can be used for the production of commercially useful quantities of the encoded polypeptide.

In yet another aspect, the present invention provides a fusion protein comprising a polypeptide according to the first aspect fused to at least one other polypeptide sequence.

In a preferred embodiment of this aspect, the at least one other polypeptide is selected from the group consisting of: a polypeptide that enhances the stability of the polypeptide of the first or the second aspect, and a polypeptide that assists in the purification of the fusion protein.

In another aspect, the present invention provides a polynucleotide encoding the fusion protein.

The present invention also provides a composition for hydrolysing an organophosphate molecule, the composition comprising a polypeptide produced by the method of the third aspect of the present invention and one or more acceptable carriers.

In a still further aspect, the present invention provides a composition for hydrolysing an organophosphate molecule, the composition comprising the host cell of the present invention and one or more acceptable carriers.

It will be appreciated that in preferred embodiments the polypeptide can be used to hydrolyse organophosphates in a sample. For instance, after a crop has been sprayed with an organophosphate pesticide, the organophosphate residue can be hydrolysed from seeds, fruits and vegetables before human consumption. Similarly, organophosphate contaminated soil or water can be treated with a polypeptide of the second aspect of the present invention.

Unless defined otherwise, all technical and scientific terms used herein have the same meaning as commonly understood by one of ordinary skill in the art (e.g. in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

Organophosphates are synthetic organophosphorus esters and related compounds such as phosphoramidates. They have the general formula (RR′X)P═O or (RR′X)P═S, where R and R′ are short-chain groups. For insecticidal organophosphates X is a good leaving group, which is a requirement for the irreversible inhibition of acetylcholinesterase. The polypeptides of the present invention hydrolyse the phosphoester bonds of organophosphates.

Although well known for their use as pesticides, organophosphates have also been used as nerve gases against mammals. Accordingly, it is envisaged that the polypeptide of the present invention will also be useful for hydrolysis of organophosphates which are not pesticides.

By “substantially purified” we mean a polypeptide that has been separated from the lipids, nucleic acids, other polypeptides, and other contaminating molecules with which it is associated in its native state.

The % identity of a polypeptide is determined by FASTA (Pearson and Lipman, 1988) analysis (GCG program) using the default settings and a query sequence of at least 50 amino acids in length, and whereby the FASTA analysis aligns the two sequences over a region of at least 50 amino acids. More preferably, the FASTA analysis aligns the two sequences over a region of 100 amino acids.

OPDA activity as used herein refers to the ability of the enzyme to hydrolase a organophosphate molecule. OPDA activity may be determined by, for example, assaying culture supernatant containing secreted OPDA for activity against an organophosphate molecule. The organophosphate molecule may be selected from the group consisting of: coumaphos, coroxon, paraoxon, parathion, parathion-methyl, phosmet, fenthion, diazinon, chlorpyrifos, and dMUP. More preferably, the organophosphate is phosmet or fenthion.

OPDA activity, such as activity against coumaphos, in for example, supernatant, may be compared to that in the cell fraction, or to the supernatant fraction obtained from a cell expressing a control, such as an unmodified OPDA.

Polynucleotides of the invention comprise nucleic acid sequences encoding the polypeptides of the invention. Polynucleotides of the invention may comprise DNA or RNA. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

It will be understood by a skilled person that numerous different polynucleotides can encode the same polypeptide as a result of the degeneracy of the genetic code.

Polynucleotides of the invention can be incorporated into a recombinant replicable vector. The vector may be used to replicate the nucleic acid in a compatible host cell. Thus, in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as Brevibacillus.

Preferably, a polynucleotide of the invention in a vector is operably linked to a regulatory sequence that is capable of providing for the expression of the coding sequence by the host cell, i.e. the vector is an expression vector. The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

Such vectors may be transformed or transfected into a suitable host cell as described above to provide for expression of a polypeptide of the invention. This process may comprise culturing a host cell transformed with an expression vector as described above under conditions to provide for expression by the vector of a coding sequence encoding the polypeptides, and optionally recovering the expressed polypeptides.

The vectors may be, for example, plasmid vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used in vitro, for example to transfect or transform a host cell.

Promoters/enhancers and other expression regulation signals may be selected to be compatible with the host cell for which the expression vector is designed. For example; prokaryotic promoters may be used, in particular those suitable for use in Brevibacillus strains (such as Bacillus brevis and Bacillus choshinensis). A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in E. coli (Raibaud et al., Ann. Rev. Genet., 18: 173, 1984). In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter (U.S. Pat. No. 4,551,433). For example, the tac promoter is a hybrid trp-lac promoter comprised of both trp promoter and lac operon sequences that is regulated by the lac repressor (Amann et al., Gene, 25: 167, 1983; de Boer et al., Proc. Natl. Acad. Sci. USA, 80: 21, 1983). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes.

Phage promoters may also be used, for example lambda. These promoters are readily available in the art.

Oligonucleotides and/or polynucleotides of the present invention may selectively hybridise to the sequence set out in SEQ ID NO:29 under high stringency. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5 × Denhardt's solution, sonicated salmon sperm DNA (50 g/ml), 0.1% SDS and 10% dextran sulfate at 42° C. in 0.2×SSC and 0.1% SDS.

Vectors and polynucleotides of the invention may be introduced into host cells for the purpose of replicating the vectors/polynucleotides and/or expressing the polypeptides of the invention encoded by the polynucleotides of the invention. Suitable host cells include prokaryotes such as Brevibacillus.

Suitable host cells to transform include any cell that can be transformed with a polynucleotide of the present invention. Host cells can be either untransformed cells or cells that are already transformed with at least one nucleic acid molecule (e.g., nucleic acid molecules encoding one or more proteins of the present invention). Host cells of the present invention can be capable of producing such proteins after being transformed with at least one nucleic acid molecule of the present invention. Host cells of the present invention can be any cell capable of producing at least one protein of the present invention.

Vectors/polynucleotides of the invention may be introduced into suitable host cells using a variety of techniques known in the art, such as transfection, transformation and electroporation. More preferred host cells are selected from the Brevibacillus cluster comprising the following species, namely, Bacillus brevis, Bacillus agri, Bacillus centrosporus, Bacillus choshinensis, Bacillus parabrevis, Bacillus reuszeri, Bacillus formosus, Bacillus borstelensis, Bacillus laterosporus, and Bacillus thermoruber. Even more preferred host cells are Bacillus brevis and Bacillus choshinensis.

Host cells of the present invention can be cultured in conventional fermentation bioreactors. The host cells can be cultured by any fermentation process which includes, but is not limited to, batch, fed-batch, cell recycle, and continuous fermentation. Preferably, host cells of the present invention are grown by batch or fed-batch fermentation processes.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell.

Host cells comprising polynucleotides of the invention may be used to express polypeptides of the invention. Host cells may be cultured under suitable conditions which allow expression of the polypeptide produced according to the invention. Expression of the polypeptides of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Polypeptides of the invention can be collected from the supernatant of cultures of host cells expressing OpdA of the invention.

Purification of polypeptides may optionally be performed using well known techniques such as affinity chromatography, including immunoaffinity chromatography, ion-exchange chromatography and the like, or affinity chromatography systems based on fusion protein sequences such as those known in the art.

Recombinant DNA technologies can be used to improve expression of polypeptide molecules by manipulating, for example, the number of copies of the polynucleotide within a host cell, the efficiency with which those polynucleotides molecules are transcribed and the efficiency with which the resultant transcripts are translated. Recombinant techniques useful for increasing the expression of polynucleotide molecules of the present invention include, but are not limited to, operatively linking polynucleotide molecules to high-copy number plasmids, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts. The activity of an expressed recombinant protein of the present invention may be improved by fragmenting, modifying, or derivatizing polynucleotide molecules encoding such a protein.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the invention.

Any discussion of documents, acts, materials, devices, articles or the like which has been included in the present specification is solely for the purpose of providing a context for the present invention. It is not to be taken as an admission that any or all of these matters form part of the prior art base or were common general knowledge in Australia in the field relevant to the present invention

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the nature of the present invention may be more clearly understood preferred forms thereof will now be described by reference to the following non-limiting Examples.

### EXAMPLES

Expression of OpdA

The amino acid sequence of OpdA including the native signal peptide is shown in SEQ ID NO. 30. In this sequence the signal peptide is residues 1 to 28 and the sequence is set out below. Examination of this sequence shows that it possesses the distinctive twin arginine motif of the TAT system which implies that OpdA is folded prior to secretion and cleavage of the signal peptide. Therefore it is unlikely that OpdA can be secreted through the Sec system.

MQTRRDALKSAAAITLLGGLAGCASMAR (SEQ ID No:31)

(The signal peptide of OpdA with the TAT motif underlined.)

In an effort to obtain higher yields of OpdA, expression of the protein was attempted using a Brevibacillus expression system developed by Higeta Shoya Co. Ltd. In using this system the native signal peptide was deleted and replaced with the following sequence:

MKKRRVVNSVLLLLLLASALALTVAPMAFA|AGS (SEQ ID No:32)

(| indicates SpaseI-cleavage site)

Attempts using this expression system were unsuccessful.

In an effort to understand this failure it was noted that while the modified signal peptide possessed the “twin arginine” motif of the TAT system it lacked a positively charged residue near the cleavage site which Tjalsma et al., (2000) had found acts as a Sec avoidance signal in Bacillus subtilis. In an effort to determine whether expression could be achieved by incorporation of a Sec avoidance signal, the signal peptide was modified to include a positively-charged residue and a “Bacillus” type signal peptidase (SPaseI) cleavage site.

Two complementary oligos (NCMO25′, 5′GTTCAGCCCATGGCTAAAGCTGCAGAGCACGGATCCGATC (SEQ ID No:33) and NCMO23′, 5′GATCGGATCCGTGCTCTGCAGCTTTAGCCATGGGCTGAAC (SEQ ID No:34)) containing an NcoI site and one end (underlined) and a BamHI site at the other end (bold-type) were designed to alter the signal peptide to that shown below.

(SEQ ID No:35)

(a) MKKRRVVNSVLLLLLLASALALTVAPMAFA|AGS

(SEQ ID No:36)

(b) MKKRRVVNSVLLLLLLASALALTVAPMAKA|AEH

The amino acid sequence of the original signal peptide in pNCMO2 (a) and the altered signal peptide in pNC(mod) (b).

The two oligos were dissolved in TE buffer to 44 nmol/ml. Ten microlitres of each were incubated for 10 minutes at 95° C. and then cooled slowly to room temperature. This would melt the two complimentary bands together. This was then digested with NcoI and BamHI. The plasmid pNCMO2 was digested with NcoI and BamHI and extracted from a 1% agarose gel using the QIAquick PCR purification kit (QIAgen). The digested pNCMO2 and digested oligos were ligated overnight and transformed the following day into E. coli DH10β. All transformations in E. coli were performed according to the revised Hanahan method (Sambrook et al., 1989). Transformants were selected on LB agar plates (10 g/l tryptone, 5 g/l Yeast Extract, 2.5 g/l NaCl) containing 100 μg/ml ampicillin. One transformant containing an altered signal peptide (as determined by DNA sequencing) was selected and designated pNC(mod).

Construction of pNC(mod)-opdA

The plasmid pNC(mod) was digested with BamHI and EcoRI and excised from a 1% agarose gel using the QIAquick PCR purification kit (QIAgen). The plasmid pGAfull was digested with BamHI and EcoRI and the 1 kb opdA containing fragment was excised from a 1% agarose gel using the QIAquick PCR purification kit and ligated overnight with the extracted pNC(mod) fragment. The ligation mixture was transformed into E. coli CC118 (araD139, Δ(ara, leu)7697, ΔlacX74, phoAΔ20, galE, galK, thi, rpsE, rpoB, argEₐₘₗ, recA1; Manoil & Beckwith, 1985) and transformants selected on LB plates containing ampicillin (100 μg/ml). The opdA expression in pNC(mod)-opdA is run from a lacZ promoter and there is leaky expression from lac promoters in some E. coli strains. However, no expression from lac promoters occurs in E. coli CC118 due to deletion of the lac operon. Transformation into E. coli DH10β resulted in point mutations in opdA, resulting in the production of a non-functional protein. This is probably due to an inability of the signal peptide to target the TAT secretion system and avoid the Sec system in E. coli.

Expression of opdA in Brevibacillus choshinensis

The plasmid pNC(mod)-opdA was transformed into Brevibacillus choshinensis. HPD31 (Takagi et al., 1989) using the Tris-PEG method of Udaka & Yamagata (1993) and transformants selected on BTY medium (glucose, 1.0%; tryptone, 2.0%; Yeast Extract, 0.5%; FeSO₄.7H₂O, 0.001%; MnSO₄.4H₂O, 0.001%; ZnSO₄.7H₂O, 0.001%) with 20 mM MgCl₂ and 50 μg/ml neomycin at 28° C. One transformant was picked and grown up for further analysis. The plasmid contained in this transformant was extracted and examined and shown to be pNC(mod)-opdA B. choshinensis pNC(mod)-opdA was grown to mid-log phase and induced with 1 mM IPTG at 28° C. for 24 hours in BTY medium with 50 μg/ml neomycin. The culture supernatant and cells were separated by centrifugation (7000 g, 10 minutes). The cell pellet was resuspended in 2 ml 50 mM Tris-HCl pH7.5 and sonicated.

Tris-PEG Method of Transformation

B. choshinensis was streaked onto BTY medium and grown at 37° C. for 2 days. A loopful of growth was then used to inoculate 5 ml of BTY medium and grown overnight at 37° C. The overnight culture was diluted 100-fold in 5 ml of the same medium and incubated at 37° C. for 5 hours. The cells were then pelleted by centrifugation (4000 g, 5 minutes) and washed with 5 ml of 50 mM Tris-HCl pH7.5. The pellet was finally resuspended in 5 ml of 50 mM Tris-HCl pH8.5 and incubated for 30 minutes at 37° C. After this incubation the cell pellet was washed with 1 ml of MTP. MTP was prepared as follows:

| | | |
|---|---|---|
| 0.1 M Sodium Maleate pH 6.5 | 20 | ml |
| Phosphate Buffer(7% w/v K₂HPO₄/2.5% w/v KH₂PO₄) | 10 | ml |
| H₂O | 18 | ml |
| Autoclave and then add: | | |
| 1 M MgCl₂ (filter-sterilised) | 2 | ml |
| BTY | 50 | ml |

Plasmid DNA was added to the cell suspension, after which 1.5 ml of a PEG solution (40 g PEG8000, 20 ml 0.1 M Sodium maleate pH6.5 and H₂0 to 100 ml) was added and the mixture incubated at room temperature for 2 minutes. MTP (5 ml) was added and mixed well. The cells were collected by centrifugation (4000 g, 10 minutes at room temperature). The cells were then resuspended in 1 ml of BTY with 20 mM MgCl₂ and incubated at 30° C. for 2.5 hours with moderate shaking.

Checking for Expression

Both the supernatant and the cell extracts were examined for coumaphos hydrolytic activity and SDS-PAGE. No coumaphos hydrolytic activity could be detected in B. choshinensis cells in the absence of pNC(mod)-opdA. The majority of the coumaphos hydrolytic activity was secreted into the culture supernatant (Table 2). The specific activity of the supernatant was 5.09 μmol/min/mg protein), which is close to that of purified OpdA (8.0 μmol min/mg protein), suggesting that OpdA in the supernatant is relatively pure.

TABLE 2
The percentage coumaphos hydrolytic activity
in B. choshinensis with various plasmids.

| Strain | Supernatant | Cells |
|---|---|---|
| B. choshinensis | 64.8 ± 7.6 | 35.2 ± 4.2 |
| pNC(mod)-opdA | | |
| B. choshinensis | nd¹ | nd |
| pNC(mod) | | |

nd¹ = not detected

The Signal Peptide Allows Secretion of Active Cytoplasmic Proteins

In general, cytoplasmic proteins readily fold in the cytoplasm of heterologous hosts. Therefore it would be predicted that this group of proteins may not be readily secreted by the Sec system β-galactosidase is a classic example of this. This particular enzyme from E. coli is a large protein of 120 kDa and known to not pass through the cytoplasmic membrane when attached to a Sec-type secretion peptide (Manoil & Beckwith, 1985; Bassford et al., 1979). Therefore, this protein was chosen to examine the secretion of active cytoplasmic proteins by this Brevibacillus system.

The lacZ gene was amplified by PCR using pEM32m as a template. The primers used were lac5c (5′CATGTCGACATGGATCCCGTCGTT) (SEQ ID No:37) and lac3b (5′CATGAATTCTTATTTTTGAACTGGTAA) (SEQ ID No:38) containing SalI and EcoRI sites, respectively. The PCR was performed using the Pfu Turbo DNA polymerase from Stratagene, according to the manufacturer's instructions. The 3 kb PCR product was purified using the QIAquick PCR purification kit and ligated with pGEM T Easy (Promega) overnight. The ligation mixture was then transformed into E. coli DH10β and transformants selected on LB plates containing ampicillin (100 μg/ml), X-Gal (5-bromo-4-chloro-3-indolyl β-D galactoside, 40 μg/ml) and IPTG (40 μg/ml). It was noted that there were some intense blue colonies on the plates. One was examined and shown to contain the 3 kb PCR product encoding β-galactosidase. This plasmid was called pGEMlac and demonstrates the presence of a functional β-galactosidase enzyme. When this insert was contained in the opposite orientation, no β-galactosidase activity was generated (Table 3).

TABLE 3
The β-galactosidase activity in E. coli
DH10β cells with various constructs.

| | β-galactosidase activity |
|---|---|
| Strain | (nmol/min/μg protein) |
| E. coli DH10β pGEMlac2¹ | 0.0054 ± 0.0009 |
| E. coli DH10β pGEMlac5² | 11.001 ± 0.007 |

¹lacZ is contained in the opposite orientation to lac promoter
²lacZ is contained in the same orientation as the lac promoter

The SalI-EcoRI fragment containing β-galactosidase was ligated with similarly-digested pNC(mod). The ligation mix was transformed into E. coliCC118 and transformants selected on LB plates containing ampicillin (100 μg/ml). Several transformants were picked and examined for inserts. One was chosen and shown to contain the 3 kb SalI-EcoRI fragment. This plasmid was designated pNC(mod)-lac. This plasmid was then transformed into Brevibacillus and transformants selected on BTY medium with 50 μg/ml neomycin at 30° C. One colony was picked after two days growth and examined for the production of β-galactosidase when induced with IPTG. The culture was pelleted by centrifugation (7000&, 10 minutes) and the cell pellet resuspended in 50 mM Tris-HCl pH7.5, after which it was sonicated. Both the cell extract and the culture supernatant were examined for -galactosidase activity. The majority of the -galactosidase activity was contained in the supernatant (Table 4). Brevibacillus choshinensis does not possess any intrinsic β-galactosidase activity.

TABLE 4
The percentage β-galactosidase activity in
B. choshinensis cells with various plasmids.

| Strain | Supernatant | Cells |
|---|---|---|
| B. choshinensis | 98.2 ± 6.0 | 1.80 ± 0.17 |
| pNC(mod)-lac | | |
| B. choshinensis | nd¹ | nd |
| pNC(mod) | | |

¹not detected

This secretion system was tested on another cytoplasmic protein, that is also a phosphotriesterase. Horne et al. (2002b) identified the protein HocA from a Pseudomonas monteilli strain that is capable of hydrolysing organophosphates. This protein is a 20 kDa cytoplasmic protein unrelated in sequence and reaction mechanism to OpdA. The hocA gene was amplified by PCR using the upstream and downstream oligonucleotide primers, hoc5, 5′GTCTAAGGATCCATGAAAGAAGAACTAAAAACC, (SEQ ID No:39) and hoc3, 5′GTCTAAAAGCTTTTACCAGTTTAGCTTTAG, (SEQ ID No:40) with BamHI and HindIII restriction sites (underlined), respectively, and the template pBSRK7(1) as a template. The PCR product was digested with BamHI and HindIII and cloned into similarly-digested pK18. The ligation was transformed into E. coli DH10β. One transformant was chosen and shown by sequence analysis to have a correct hocA gene. This clone was designated pKhoc2. The 501 bp hocA-containing BamHI-HindIII fragment of pKhoc2 was then cloned into pNC(mod) and transformed into E. coli CC118. One clone was selected that possessed hocA and this clone was designated pNC(mod)-hoc. The plasmid pNC(mod)-hoc was transformed into Brevibacillus choshinensis with transformants selected on BTY medium with 50 μg/ml neomycin at 30° C. One colony was picked after two days growth and grown in 50 ml BTY medium to mid-log phase and then induced with 1 mM IPTG at 28° C. for 24 hours. The culture supernatant and cells were separated by centrifugation (7000 g, 10 minutes). The cell pellet was resuspended in 50 mM Tris-HCl pH7.5 and sonicated. The coumaphos hydrolytic activity in the cell extract and in the culture supernatant was determined. Table 5 shows the relative amounts of activity. The majority of the protein was secreted in an active form into the culture supernatant.

TABLE 5
The percentage coumaphos hydrolytic activity
in B. choshinensis with various plasmids.

| Strain | Supernatant | Cells |
|---|---|---|
| B. choshinensis | nd¹ | nd |
| pNC(mod) | | |
| B. choshinensis | 75.1 ± 6.8 | 24.9 ± 0.9 |
| pNC(mod)-hoc | | |

¹not detected

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Bassford, Jr., P. J., T. J. Silhavy & J. R. Beckwith. 1985. Use of gene fusion to study secretion of maltose-binding protein into Escherichia coli periplasm. J. Bacteriol. 139: 19-31.
Berks, B. C. 1996. A common export pathway for proteins binding complex redox cofactors? Mol. Microbiol. 22: 393-404.
Harcourt, R. L., I. Horne, T. D. Sutherland, B. D. Hammock, R. J. Russell & J. G. Oakeshott. 2002. Development of a simple and sensitive fluorimetric method for the isolation of cournaphos-hydrolysing bacteria from the environment. Lett. Appl. Microbiol. 34: 263-268.
Horne, I, T. D. Sutherland, R. L. Harcourt, R. J. Russell & J. G. Oakeshott. 2002a. Identification of an opd (organophosphate degradation) gene in an Agrobacterium isolate. Appl. Environ. Microbiol. 68: 3371-3376
Horne, I., T. D. Sutherland, J. G. Oakeshott & R. J. Russell. 2002b. Cloning and expression of the phosphotriesterase gene, hocA, from Pseudomonas monteilli C11. Microbiol. Accepted for publication.
Machowski, E. E., R. A. McAdam, K. M. Derbyshire & V. Mizrahi. 2000. Construction and application of mycobacterial reporter transposons. Gene 253: 67-75.
Manoil, C. & J. Beckwith. 1985. TriphoA: a transposon probe for protein export signals. Proc. Natl. Acad. Sci. USA 82: 8129-8133.
Miyauchi, A., M. Ozawa, M. Mizukami, K. Yashiro, S. Ebisu, T. Tojo, T. Fujii & H. Takagi. 1999. Structural conversion from non-native to native form of recombinant human epidermal growth factor by Brevibacillus choshinensis. Biosci. Biotechnol. Biochem. 63: 1965-1969.
Morris, S. H. & C. C. Adley. 2001. Irish public perceptions and attitudes to modern biotechnology: an overview with a focus on GM foods. Trends in Biotechnol. 19: 43-48.
Nagarajan, V. 1990. System for secretion of heterologous proteins in Bacillus subtilis. Methods Enzymol. 185: 214-228.
Neu, H. C. & L. A. Heppel. 1965. The release of enzymes from Escherichia coli by osmotic shock and during the formation of spheroplasts. J. Biol. Chem. 240: 3685-3692.
Rowland, S. S., J. J. Zulty, M. Sathyamoorthy, B. M. Pogell & M. K. Speedie. 1992. The effect of signal sequences on the efficiency of secretion of a heterologous phosphotriesterase by Streptomyces lividans. Appl. Microbiol. Biotechnol. 38: 94-100.
Sambrook, J., E. F. Fritsch & T. Maniatis. 1989. Molecular cloning—A Laboratory Manual. 2^{nd} Ed. Cold Spring Harbour Laboratory Press, USA.
Simon, R., U. Priefer & A. Puhler. 1983. A broad host range mobilization system for in vivo genetic engineering: transposon mutagenesis in Gram-negative bacteria. Bio/Technol. 1: 784-791.
Steiert, J. G., B. M. Pogell, M. K. Speedie & J. Laredo. 1989. A gene coding for a membrane-bound hydrolase is expressed as a secreted-soluble enzyme in Streptomyces lividans. Bio/Technol. &: 65-68.
Takagi, H., K. Kadowski & S. Udaka. 1989. Screening and characterization of protein hyper-producing bacteria without detectable exoprotease activity. Agric. Biol. Chem. 53: 691-699.
Tjalsma, H., A. Bolhuis, J. D. Jongbloed, S. Bron & J. M. van Dijl. 2000. Signal peptide-dependent protein transport in Bacillus subtilis: a genome-based survey of the secretome. Microbiol. Molecul. Biol. Rev. 64: 515-547.
Udaka, S. & H. Yamagata. 1993. High-level secretion of heterologous proteins by Bacillus brevis. Meth. Enzymol. 217: 23-33.

1. A recombinant polynucleotide, the polynucleotide comprising a first and a second sequence, the first sequence encoding a signal peptide comprising a TAT signal and a Sec avoidance signal and the second sequence encoding a heterologous protein, wherein the sequence of the signal peptide is
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

2. A recombinant polynucleotide according to claim 1 wherein X₁ is a sequence of 0 to 5 amino acids, and is preferably 0.

3. A recombinant polynucleotide according to claim 1 wherein X₂ is a sequence of 0 or 1 amino acid, preferably 0.

4. A recombinant polynucleotide according to claim 1 wherein X₃ is a sequence of 0 to 5 amino acids, preferably 0.

5. A recombinant polynucleotide according to claim 1 wherein X₄ is a sequence of at least 20 amino acids of which at least 18 are hydrophobic amino acids.

6. A recombinant polynucleotide according to claim 1 wherein X₄ is 23 amino acids.

7. A recombinant polynucleotide according to claim 1 wherein the sequence of the signal peptide is MKKRRVVNSVLLLLLLASALALTVAPMAKA (SEQ ID NO:1).

8. A signal peptide, the signal having the sequence
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.

9. A signal peptide according to claim 8 wherein X₁ is a sequence of 0 to 5 amino acids, and is preferably 0.

10. A signal peptide according to claim 8 wherein X₂ is a sequence of 0 to 1 amino acid, preferably 0.

11. A signal peptide according to claim 8 wherein X₃ is a sequence of 0 to 5 amino acids, preferably 0.

12. A signal peptide according to claim 8 wherein X₄ is a sequence of at least 20 amino acids of which at least 18 are hydrophobic amino acids.

13. A signal peptide according to claim 8 wherein X₄ is 23 amino acids.

14. A signal peptide according to claim 8 wherein the sequence of the signal peptide is MKKRRVVNSVLLLLLLASALALTVAPMAKA (SEQ ID NO 1).

15. A method of producing a heterologous polypeptide from a host cell comprising a TAT translocation system, the method comprising:
(i) transforming the host cell with a DNA sequence encoding the heterologous polypeptide and a signal peptide wherein the signal peptide comprises a TAT signal and a Sec avoidance signal wherein the sequence of the signal peptide is
M-X₁—K/R—X₂—K/R—X₃—RR—X₄—K/R-A
in which
X₁ is a sequence of 0 to 10 amino acids;
X₂ is a sequence of 0 to 3 amino acids;
X₃ is a sequence of 0 to 10 amino acids; and
X₄ is a sequence of 15 to 24 amino acids in which at least 75% up to about 90% of the residues are hydrophobic.
(ii) culturing the host cell under conditions which allow expression of the heterologous polypeptide; and
(iii) recovering the heterologous polypeptide secreted from the host cell via the TAT translocation system.

16. A method according to claim 15 wherein X₁ is sequence of 0 to 5 amino acids, and is preferably 0.

17. A method according to claim 15 wherein X₂ is a sequence of 0 or 1 amino acid, preferably 0.

18. A method according to claim 15 wherein X₃ is a sequence of 0 to 5 amino acids, preferably 0.

19. A method according to claim 15 wherein X₄ is a sequence of at least 20 amino acids of which at least 18 are hydrophobic amino acids.

20. A method according to claim 15 wherein X₄ is 23 amino acids.

21. A method according to claim 15 wherein the sequence of the signal peptide is MKKRRVVNSVLLLLLLASALALTVAPMAKA (SEQ ID NO:1).

22. A method according to claim 15 wherein the host cell is Bacillus sp.

23. A method according to claim 22 wherein the host cell is selected from the group consisting of Bacillus choshinensis, Bacillus brevis, Bacillus subtilis, Bacillus licheniformis, and Bacillus megatorium.

24. A method according to claim 22 wherein the host cell is Bacillus choshinensis.

25. A method according to claim 15 wherein the heterologous polypeptide is a polypeptide which readily folds in the cytoplasm.

26. A method according to claim 15 wherein the polynucleotide encoding the mature polypeptide has a sequence selected from:
(i) a sequence of nucleotides shown in SEQ ID NO:29 from nucleotide 85 to 1155;
(ii) a sequence that hybridises to SEQ ID NO:29 from nucleotide 85 to 1155 under conditions of high stringency;
(iii) a sequence which is greater than 90% identical to SEQ ID NO:29 from nucleotide 85 to 1155; and
(iv) a sequence that encodes the amino acid sequence provided in SEQ ID NO:30 from residue 29 to 384.

27. A method according to claim 15 wherein the mature heterologous polypeptide comprises the sequence provided in SEQ ID NO:30 from residue 29 to 384; or a polypeptide which is greater than 90% identical to the sequence provided in SEQ ID NO:30.

28. A substantially purified polypeptide produced according to the method of claim 15.

29. A vector comprising the recombinant polynucleotide according to claim 1.

30. A host cell comprising the recombinant polynucleotide according to claim 1.


SEQUENCE LISTING (40 sequences)

SEQ ID NO: 1 - protein, 30 aa, Artificial Sequence
  signal sequence
MKKRRVVNSVLLLLLLASALALTVAPMAKA

SEQ ID NO: 2 - protein, 46 aa, Artificial Sequence
  signal sequence
MEARMTGRRKVTRRDAMADAARAVGVACLGGFSLAALVRTASPVDA

SEQ ID NO: 3 - protein, 41 aa, Artificial Sequence
  signal sequence
MSRSAKPQNGRRRFLRDVVRTAGGLAAVGVALGLQQQTARA

SEQ ID NO: 4 - protein, 27 aa, Artificial Sequence
  signal sequence
MTWSRRQFLTGVGVLAAVSGTAGRVVA

SEQ ID NO: 5 - protein, 27 aa, Artificial Sequence
  signal sequence
MDRRRFLTLLGSAGLTATVATAGTAKA

SEQ ID NO: 6 - protein, 37 aa, Artificial Sequence
  signal sequence
MSEKDKMITRRDALRNIAVVVGSVATTTMMGVGVADA

SEQ ID NO: 7 - protein, 34 aa, Artificial Sequence
  signal sequence
MQIVNLTRRGFLKAACVVTGGALISIRMTGKAVA

SEQ ID NO: 8 - protein, 45 aa, Artificial Sequence
  signal sequence
MNNEETFYQAMRRQGVTRRSFLKYCSLAATSLGLGAGMAPKIAWA

SEQ ID NO: 9 - protein, 48 aa, Artificial Sequence
  signal sequence
MSTGTTNLVRTLDSMDFLKMDRRTFMKAVSALGATAFLGTYQTEIVNA

SEQ ID NO: 10 - protein, 50 aa, Artificial Sequence
  signal peptide
MKCYIGRGKNQVEERLERRGVSRRDFMKFCTAVAVAMGMGPAFAPKVAEA

SEQ ID NO: 11 - protein, 26 aa, Artificial Sequence
  signal sequence
MNRRNFIKAASCGALLTGALPSVSHA

SEQ ID NO: 12 - protein, 44 aa, Artificial Sequence
  signal sequence
MSHADEHAGDHGATRRDFLYYATAGAGTVAAGAAAWTLVNQMNP

SEQ ID NO: 13 - protein, 44 aa, Artificial Sequence
  signal sequence
MTQISGSPDVPDLGRRQFMNLLTFGTITGVAAGALYPAVKYLIP

SEQ ID NO: 14 - protein, 32 aa, Artificial Sequence
  signal sequence
MDRRTFLRLYLLVGAAIAVAPVIKPALDYVGY

SEQ ID NO: 15 - protein, 42 aa, Artificial Sequence
  signal sequence
MTKLSGQELHAELSRRAFLSYTAAVGALGLCGTSLLAQGARA

SEQ ID NO: 16 - protein, 31 aa, Artificial Sequence
  signal sequence
MTLTRREFIKHSGIAAGALVVTSAAPLPAWA

SEQ ID NO: 17 - protein, 31 aa, Artificial Sequence
  signal sequence
MTISRRDLLKAQAAGIAAMAANIPLSSQAPA

SEQ ID NO: 18 - protein, 32 aa, Artificial Sequence
  signal sequence
MSEALSGRGNDRRKFLKMSALAGVAGVSQAVG

SEQ ID NO: 19 - protein, 45 aa, Artificial Sequence
  signal sequence
MKTKIPDAVLAAEVSRRGLVKTTAIGGLAMASSALTLPFSRIAHA

SEQ ID NO: 20 - protein, 35 aa, Artificial Sequence
  signal sequence
MSNFNQISRRDFVKASSAGAALAVSNLTLPFNVMA

SEQ ID NO: 21 - protein, 30 aa, Artificial Sequence
  signal sequence
MSISRRSFLQGVGIGCSACALGAFPPGALA

SEQ ID NO: 22 - protein, 37 aa, Artificial Sequence
  signal sequence
MKTVLPSVPETVRLSRRGFLVQAGTITCSVAFGSVPA

SEQ ID NO: 23 - protein, 44 aa, Artificial Sequence
  signal sequence
MGRLNRFRLGKDGRREQASLSRRGFLVTSLGAGVMFGFARPSSA

SEQ ID NO: 24 - protein, 50 aa, Artificial Sequence
  signal sequence
MSDKDSKNTPQVPEKLGLSRRGFLGASAVTGAAVAATALGGAVMTRESWA

SEQ ID NO: 25 - protein, 32 aa, Artificial Sequence
  signal sequence
MESRTSRRTFVKGLAAAGVLGGLGLWRSPSWA

SEQ ID NO: 26 - protein, 27 aa, Artificial Sequence
  signal sequence
MSLSRRQFIQASGIALCAGAVPLKASA

SEQ ID NO: 27 - protein, 30 aa, Artificial Sequence
  signal sequence
MTLNRRDFIKTSGAAVAAVGILGFPHLAFG

SEQ ID NO: 28 - protein, 45 aa, Artificial Sequence
  signal sequence
MTDSRANRADATRGVASVSRRRFLAGAGLTAGAIALSSMSTSASA

SEQ ID NO: 29 - DNA, 1155 bp, Agrobacterium radiobacter
atgcaaacgagaagagatgcacttaagtctgcggccgcaataactctgctcggcggcttggctgggtgtgcaagcatggcccgaccaatcggtacaggcgatctgattaatactgttcgcggccccattccagtttcggaagcgggcttcacactgacccatgagcatatctgcggcagttcggcgggattcctacgtgcgtggccggagtttttcggtagccgcaaagctctagcggaaaaggctgtgagaggattacgccatgccagatcggctggcgtgcaaaccatcgtcgatgtgtcgactttcgatatcggtcgtgacgtccgtttattggccgaagtttcgcgggccgccgacgtgcatatcgtggcggcgactggcttatggttcgacccgccactttcaatgcgaatgcgcagcgtcgaagaactgacccagttcttcctgcgtgaaatccaacatggcatcgaagacaccggtattagggcgggcattatcaaggtcgcgaccacagggaaggcgaccccctttcaagagttggtgttaaaggcagccgcgcgggccagcttggccaccggtgttccggtaaccactcacacgtcagcaagtcagcgcgatggcgagcagcaggcagccatatttgaatccgaaggtttgagcccctcacgggtttgtatcggtcacagcgatgatactgacgatttgagctacctaaccggcctcgctgcgcgcggatacctcgtcggtttagatcgcatgccgtacagtgcgattggtctagaaggcaatgcgagtgcattagcgctctttggtactcggtcgtggcaaacaagggctctcttgatcaaggcgctcatcgaccgaggctacaaggatcgaatcctcgtctcccatgactggctgttcgggttttcgagctatgtcacgaacatcatggacgtaatggatcgcataaacccagatggaatggccttcgtccctctgagagtgatcccattcctacgagagaagggcgtcccgccggaaacgctagcaggcgtaaccgtggccaatcccgcgcggttcttgtcaccgaccgtgcgggccgtcgtgacacgatctgaaacttcccgccctgccgcgcctattccccgtcaagataccgaacgatga

SEQ ID NO: 30 - protein, 384 aa, Agrobacterium radiobacter
MQTRRDALKSAAAITLLGGLAGCASMARPIGTGDLINTVRGPIPVSEAGFTLTHEHICGSSAGFLRAWPEFFGSRKALAEKAVRGLRHARSAGVQTIVDVSTFDIGRDVRLLAEVSRAADVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 31 - protein, 28 aa, Artificial Sequence
  signal sequence
MQTRRDALKSAAAITLLGGLAGCASMAR

SEQ ID NO: 32 - protein, 33 aa, Artificial Sequence
  signal sequence
MKKRRVVNSVLLLLLLASALALTVAPMAFAAGS

SEQ ID NO: 33 - DNA, 40 bp, Artificial Sequence
  oligonucleotide
gttcagcccatggctaaagctgcagagcacggatccgatc

SEQ ID NO: 34 - DNA, 40 bp, Artificial Sequence
  oligonucleotide
gatcggatccgtgctctgcagctttagccatgggctgaac

SEQ ID NO: 35 - protein, 33 aa, Artificial Sequence; identical to SEQ ID NO: 32
  signal sequence

SEQ ID NO: 36 - protein, 33 aa, Artificial Sequence
  signal sequence
MKKRRVVNSVLLLLLLASALALTVAPMAKAAEH

SEQ ID NO: 37 - DNA, 24 bp, Artificial Sequence
  oligonucleotide
catgtcgacatggatcccgtcgtt

SEQ ID NO: 38 - DNA, 27 bp, Artificial Sequence
  oligonucleotide
catgaattcttatttttgaactggtaa

SEQ ID NO: 39 - DNA, 33 bp, Artificial Sequence
  oligonucleotide
gtctaaggatccatgaaagaagaactaaaaacc

SEQ ID NO: 40 - DNA, 30 bp, Artificial Sequence
  oligonucleotide
gtctaaaagcttttaccagtttagctttag